(19) United States
(12) Patent Application Publication
(10) Pub. No.: US 2022/0157465 A1
(43) Pub. Date: May 19, 2022

(54) SYSTEM AND METHOD FOR DETERMINING PERCEIVED RISK OF ENTERING PREMISES BASED ON KNOWLEDGE OF SYNDROMIC PROFILE OF OCCUPANTS

(71) Applicants: Neil Iyer, Palo Alto, CA (US); Charles Mandell, Boca Raton, FL (US); Arun Sobti, South Barrington, IL (US); Raj Panchal, South Barrington, IL (US)

(72) Inventors: Neil Iyer, Palo Alto, CA (US); Charles Mandell, Boca Raton, FL (US); Arun Sobti, South Barrington, IL (US); Raj Panchal, South Barrington, IL (US)

(21) Appl. No.: 17/530,993

(22) Filed: Nov. 19, 2021

Related U.S. Application Data
(60) Provisional application No. 63/116,023, filed on Nov. 19, 2020.

(51) Int. Cl. G16H 50/30; G16H 50/20; G16H 70/60

(52) U.S. Cl. CPC G16H 50/30; G16H 70/60; G16H 50/20

(57) ABSTRACT

The present disclosure relates to a system for determination of perceived risk of entering a premises, said system comprising a processor, a memory storing a set of instructions, which when executed by the processor, cause the processor to: review, based on receipt of a first set of data packets from one or more computing devices associated with a plurality of users that form part of premises, syndromic profiles of said plurality of users, said syndromic profiles being indicative of diagnostic testing status of the plurality of users for at least one medical indication; and determine, based on processing of the syndromic profiles of said plurality of users, overall risk associated with entering the premises based on comparison of overall assessment of the syndromic profiles of said plurality of users with one or more threshold levels.

### CROSS REFERENCE TO RELATED APPLICATION(S)

This application claims the benefit of Provisional U.S. Patent Application No. 63/116,023 filed Nov. 19, 2020, entitled “PERCEIVED RISK OF ENTERING PREMISES BASED UPON KNOWLEDGE OF SYNDROMIC PROFILE OF OCCUPANTS,” the entire content and disclosure of which, both express and implied, is incorporated herein by reference

### TECHNICAL FIELD

The present disclosure relates generally to the field of authentication and certification of diagnostic testing and the use of mobile devices to check upon a perceived risk prior to entering a premise. In particular, the present disclosure offers a solution that allows enterprises to operate in a relatively safer fashion, prove that they are adhering to local ordinances regarding operating guidance to control contagion as issued by local authorities. Further it offers any prospective client/customer knowledge of the “risk of infection” prior to entering the premise. Finally, this solution also accounts for the level of contagion that the prospective client/customer actually hails from and the clearance requirement of the destination location.

### BACKGROUND

Background description includes information that may be useful in understanding the present invention. It is not an admission that any of the information provided herein is prior art or relevant to the presently claimed invention, or that any publication specifically or implicitly referenced is prior art.

Any widespread infection, and especially ones that are highly contagious can completely wreck markets for entertainment, hospitality, and travel, among many other industries. The fear of getting infected prevents customers, users, and vendors from spending money on these industry vertical markets. This is true for any major epidemic that is contagious, but it is devastating for a pandemic as contagious as any of the SARS viruses.

To avoid total collapse of these industries and many others, regulations regarding occupancy, spacing and various parameter checks are enforced so that the vendors can continue operating, but within the allowable restrictions. Further, these restrictions are governed by public health regulatory organizations at every level, starting with globally from health organization (e.g WHO), country-wide (e.g. CDC), state-wide, county-wide, city-wide etc and are published through respective government websites. Even within a city, individual business may conservatively refine the restrictions of their respective city ordinance for better safety. Indeed, it becomes a valued parameter if a particular vendor in any industry shows how their enterprise is “safer” than their competition. While at a government level, restrictions are defined, individual businesses and franchises may have their own refinements for improving the safety for their incoming customers and associates.

For some jurisdictions it is not sufficient that a person has been tested within the past, say 48 hours, it is a firm statement that any person hailing from a region that has been declared as highly contagious must quarantine for a certain period prior to visiting said premises. For such cases, the premise owner must know of this requirement and hence the QR code that attests to the results and date of a syndromic diagnostic test must also include the specific geographic location where the test was taken/recorded. This implies that a global database depicting the infection rates of granular regions must be accessed. This type of a database exists in the USA and is offered through a partnership by the CDC. As this testing becomes more diffused in the population, such databases will be readily available globally. The present application presents the attributes of such a database and suggests an example of one.

There is another aspect of controlling a spread of contagious disease that requires better knowledge of access to certain regions that house a more vulnerable population. An example of this would be nursing homes. They house some of the most vulnerable members of the population therefore restrictions regarding occupancy, spacing and certifying the absence of infection on every person entering the facility is mandatory. Nursing homes are also competitive enterprises, and a significant parameter of merit is the “safety” of its clients and employees. A nursing home that can prove that through technology and processes, that his/her nursing home is safer can attract more revenue.

Lastly, one important measure of controlling a contagious epidemic is called contact tracing. This lets the authorities know of the potentially infected people that the known infected person based upon the contact history. Recently, contact tracing has become easier because of the known geo-location of phones and software that stores the numbers of people that were within a certain distance for a certain period with the infected person. Contact tracing are, however, considered intrusive and hence avoided by some users. The proposed scheme does not track the user but does requires that he/she check in before entering a location. Therefore, location is registered, but anonymized to maintain privacy.

In any kind of seasonal or pandemic contagion, there is a general requirement to enable people and the businesses to safely congregate in an indoor/outdoor public/private defined region. There is, therefore, a requirement in the art for mechanisms that enable a user to, before entering a premise, assess syndromic profile of users that are in the premise at that moment, and accordingly decide/get recommended whether or not to enter the said premise.

### SUMMARY

The present disclosure relates to a system for determination of perceived risk of entering a premises, said system comprising: a processor; a memory storing a set of instructions, which when executed by the processor, cause the processor to: review, based on receipt of a first set of data packets from one or more computing devices associated with a plurality of users that form part of premises, syndromic profiles of said plurality of users, said syndromic profiles being indicative of diagnostic testing status of the plurality of users for at least one medical indication; and determine, based on processing of the syndromic profiles of said plurality of users, overall risk associated with entering the premises based on comparison of overall assessment of the syndromic profiles of said plurality of users with one or more threshold levels. In an aspect, the at least one medical indication is selected from any or a combination of Covid-19, a viral infection/disease, and a contagious disease. In another aspect, the first set of data packets are received from the one or more computing devices associated with the plurality of users through an interface that wirelessly receives the first set of data packets from users that have published their diagnostic testing status. In yet another aspect, the one or more computing devices are selected from any or a combination of a laptop, a mobile phone, a smart phone, a wearable device, and a processing unit associated with the respective user. In an aspect, a first user of the plurality of users determine their respective diagnostic testing status through a self-administering test (SAT) kit, said testing status being authenticated by a trained medical service provider that is configured as a trusted authority (TA). In another aspect, syndromic profile of a respective user further indicates vaccination status of the respective user for the at least one medical indication. In another aspect, the syndromic profile of the respective user further indicates when the last test was done, testing history, whether the user contracted the at least one medical indication in the past, travel history of the respective user, and demographic profile of the said user. In another aspect, the first set of data packets are received from such users that have authorized their testing status to be transmitted, and/or whose testing status has been authenticated. In yet another aspect, the first set of data packets are received only from such users that are within a specific range/proximity from the processor. In an embodiment of the invention, the first set of data packets are received in response to a query sent through the processor to the plurality of users that are within defined proximity. In another embodiment, the first set of data packets are received in response to a query sent through the processor remotely to the plurality of users that are identified based on a geo-location specification mentioned in the query. In yet another aspect, the first set of data packets are received from a manager of the premises so as to represent the syndromic profiles of said plurality of users that are currently occupying the premises. In another aspect, the system establishes an enforceable restrictive access for a second user from entering the premises based on determination of the syndromic profile of the respective second user. In another aspect, the system is executable to display, on a display device, through a graphical representation means, any or a combination of the syndromic profiles of said plurality of users and/or the determined risk. In another aspect, the graphical representation is undertaken through any or a combination of a color coding scheme, or granularity region based on region's regulatory body's mandated or enforced characterization, such that when the user hovers around a pin of a desired region, the display presents the graphical representation for the desired region.

### BRIEF DESCRIPTION OF THE DRAWINGS

FIGS. 1 and 2 illustrate an exemplary representing highlighting the process and a sample of the information received upon the query by a prospective client/customer according to an embodiment.

FIG. 3 illustrates an exemplary flow diagram showing exemplary implementation of the proposed invention.

FIG. 4 illustrates an exemplary representation showing premises having a set of users/occupants.

### DETAILED DESCRIPTION

The following is a detailed description of embodiments of the disclosure depicted in the accompanying drawings. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

If the specification states a component or feature “may”, “can”, “could”, or “might” be included or have a characteristic, that particular component or feature is not required to be included or have the characteristic.

As used in the description herein and throughout the claims that follow, the meaning of “a,” “an,” and “the” includes plural reference unless the context clearly dictates otherwise. Also, as used in the description herein, the meaning of “in” includes “in” and “on”unless the context clearly dictates otherwise.

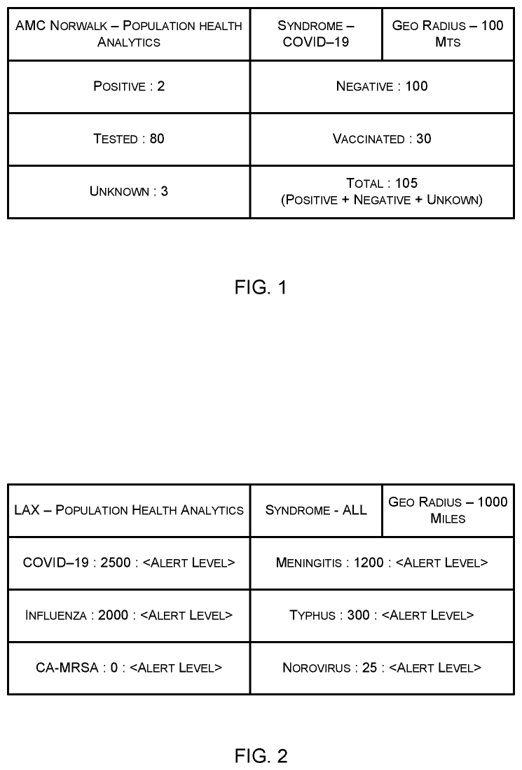

Exemplary embodiments will now be described more fully hereinafter with reference to the accompanying drawings, in which exemplary embodiments are shown. FIGS. 1 and 2 show a flow diagram highlighting the process and a sample of the information received upon the query by a prospective client/customer. These exemplary embodiments are provided only for illustrative purposes and so that this disclosure will be thorough and complete and will fully convey the scope of the invention to those of ordinary skill in the art. The invention disclosed may, however, be embodied in many different forms and should not be construed as limited to the embodiments set forth herein. Various modifications will be readily apparent to persons skilled in the art. The general principles defined herein may be applied to other embodiments and applications without departing from the spirit and scope of the invention. Moreover, all statements herein reciting embodiments of the invention, as well as specific examples thereof, are intended to encompass both structural and functional equivalents thereof. Additionally, it is intended that such equivalents include both currently known equivalents as well as equivalents developed in the future (i.e., any elements developed that perform the same function, regardless of structure). Also, the terminology and phraseology used is for the purpose of describing exemplary embodiments and should not be considered limiting. Thus, the present invention is to be accorded the widest scope encompassing numerous alternatives, modifications, and equivalents consistent with the principles and features disclosed. For purpose of clarity, details relating to technical material that is known in the technical fields related to the invention have not been described in detail so as not to unnecessarily obscure the present invention.

The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

As would be appreciated, existing systems allow users to get themselves tested for viruses such as Covid-19, and accordingly publish, get published, or report their test results through smart devices such as a wearable device or even smart phones or through applications embedded therein. In an aspect, a user can also get himself authenticated and certified by undergoing a diagnostic testing process such that the results of the testing can be obtained through telemedicine or at physical testing centres or even at home by getting a member of a diagnostic centre to test the user. The test results (also referred to as medical results or medical profile of the user) can also be securely exchanged between a patient and a medical service provider. In an exemplary aspect, the system can include an approach to receive, by a patient at a location where the patient is situated, and administer, by the patient or a care provider situated at the location of the patient, a self-administering test (herein, abbreviated as “SAT”) kit as per guidelines established for the SAT test. The administration of the SAT can occur under the supervision of a trained medical service provider with the medical service provider being located remotely. For purposes of authentication, the trained medical service provider may be considered as a trusted authority (TA). The system can further include a means to verify and authenticate the received SAT kit as being an approved and appropriate testing kit that is still within its validity date, according to instructions issued pertaining to the testing kit, either by the medical service provider, the TA or the patient. Inclusion of the TA adds to authentication of delivery and administration of the SAT.

Taking further from the above exemplary embodiments and implementations where different users/members have their diagnostic test/medical results/profile available for access to third parties (based on the level of authorization and authentication) by one or more applications that configured in/through smart devices, aspects of the present invention teach enforcement of how a user can review the medical/test/syndromic profiles of people in a defined premise/area (such as a nursing home or a restaurant or a shop or a hotel or an Airport Gate or a confined space or vehicle like an aircraft or a bus or a cab), and ensure that he/she does/does not enter into the area/premise in case the test profile is indicative of a value/profile that is below/beyond a threshold value/profile i.e., for instance, the number of people who have been tested positive or haven't got their diagnostic test done are greater than a defined number.

Aspects of the present invention therefore enable establishment of status of potentially infected/not infected users/members that are already in an establishment (i.e. physically present in a restaurant or a hotel, for instance) see FIG. 2, and offering this information to a potential user/customer/member who is currently outside the establishment/premise and queries this information in order to assess whether to enter this establishment (e.g. restaurant, bar, concert, theme park, airplane or similar) or not, or to determine if there is another similar establishment that has a lower risk profile of infected patients/area. Vendors that are stricter regarding entry and can prove this could enjoy a competitive advantage over ones that were not as strict or could not prove it.

The present disclosure enables establishments to establish an enforceable restrictive access for everyone entering the premises, identify and geo-fence areas of contamination/infection for users entering and for people staying in the premises. Although the proposed disclosure has been applied to enforce that infected people cannot enter in an exemplary manner, use of authenticated certificates can also additionally ensure compliance that proves that the probability of an infected person attempting entry is minimized. Further, the present disclosure also enables geo-fencing of potentially infectious areas as a user traverses through the establishment.

In an aspect of the present disclosure, above-mentioned exemplary implementation of verification and certification system for diagnostic medical/test/syndromic profiling of users (i.e. remote testing of users for Covid-19) can be implemented in a variety of ways.

Aspects of the present invention utilize GPS information for mobile and/or wireless system for all participants that desire to use remote access to determine status of users/clients/customers within a premise, or choose to register from a distance. Aspects of the present invention relates to a system specifically customized with the various embodiments of this invention. Yet another aspect of the present invention is to further improve upon existing portable map solution with additional attributes or characterization about chosen geolocation at any granularity. For example, when a user opens a native map application, the proposed system (configured, for instance, in the form of an executable application) may be either a native platform application or downloaded from a platform application store, Regardless, when the user executes the proposed system at any granularity, the system can be configured to display primary attributes and/or characterizations defined in various embodiments of the present invention—see FIG. 1 for an example of this information flow. The characterization may be in the form of a colour coding or the granularity region based on the region's regulatory body's mandated or enforced characterization. The attributes and characterizations in this invention relate to the syndromic and pandemic infections prevailing in the jurisdiction. When the user hovers around the pin icon of the region, the App display's the necessary information about the syndromic and pandemic infections risks levels as they apply to the granularity and scope of the region. As the granularity and scope of the selection changes from coarse down to a specific address location or a named geolocation, the syndromic and pandemic infections risks levels associated with the named geolocation are displayed to the user. For example, at a state or city level granularity, the displayed syndromic and pandemic infections risks level information can be as mandated by the state or city's government website. At a specific named geolocation, the displayed information can be retrieved from the business at the named geo location. For any business enterprise and franchise at a specific geo location general information about the business, like website, now open or not, hours of operation, current wait time etc are automatically presented to the user on the map app. The present invention enables the display of the syndromic and pandemic infections risks level for this premise/location. Additionally, depending on the user's location with respect to the selected region and/or geolocation, the information enabled and displayed can be general or privileged based on various criteria of the user's affiliation with the target region.

The proposed system can include or be operatively coupled with a relevant regulatory jurisdiction's database that relies on mapping techniques, wherein a prospective user can “pin” a certain establishment, and the proposed system can thereafter determine, using GPS information, number of people registered (in or signed in to be admitted) with the establishment. Users, through the proposed system, can also receive information describing the number of people who have been screened and or tested in the last 24-48 hours, their screening/testing status and similarly for vaccination—their status—vaccinated (within allowable time of immunity) or not. As the user navigates by zooming in or out on the map application, relevant regulatory jurisdiction's database is coupled for the purpose of retrieving the syndromic information.

Another aspect of the proposed system can include ability of a user to check-in and make a declaration regarding his/her health i.e. whether or not he has undertaken the diagnostic test or if he has previously been tested positive or when the last test was done and if the test is valid or expired, among other information that can be processed by the system in real-time, which can be done remotely by phone, or by entering this as part of the interface of the proposed system. Further, to comply with all forms of jurisdiction, the location where the last syndromic diagnostic test was conducted is encrypted into Q-R code. A global database of “infection rate” in as granular a fashion as jurisdictions desire is then referenced to ascertain if the prospective client/customer must quarantine for a certain period or is indeed able to visit the premises upon a successful syndromic diagnostic test.

In an exemplary implementation, the proposed system can be configured as an executable application, which when run by a processor can enable, for instance, merchants or vendors (such as restaurant owners) to offer desired information to potential clients/users (such as customers), and who desire to keep track of other users who have entered their premises. For instance, a restaurant A can keep a track of syndromic profile of users that are entering the said restaurant A in a manner such that a user X can review the profile of users to understand the medical profile of users that are currently there in the restaurant and then decide whether or not to enter the same. As an example, if a significant number of occupants have tested negative in the last 48 hours but hail (and were tested in) from an area of very high contagion, it may have a different impact on a prospective client/customer's desire to be present in those premises. It should be appreciated that the terms “premise”, “establishment” need not be a specific constrained physical area but can also be a vehicle like an Airplane or a bus or a train or an open area such as ground, or a road where some procession is to take place, for instance, and can therefore range from a few square feet to many miles.

In an exemplary aspect of the present invention—shown in FIG. 1, a user can, through a smart device, pin a location that can pertain to a designated area such as a restaurant or a theatre or a medical facility or a theme park, for instance, or can also enter a particular address/establishment that may wish to enter/access. Upon receipt of said location/address/pin by server/cloud of the proposed system, the system can, in real-time, extract a list of members/users within a user defined radius (called perimeter area) that form part of the desired establishment in that time instant, along with corresponding the syndromic profile of the members/users—see an exemplary sample in FIG. 2. User can then process the received information based on one or more analytics or filters using one or more parameters/attributes so that he/she can an informed decision on whether to go to enter/not go to the defined establishment. Such a attributes/parameters can, for instance, include but are not limited to, total number of people in the establishment, how many of those are Covid-negative, how many of them are positive, how many of them were positive at one time but are now negative, for how many of them the diagnostic status is not presently known, how many of them have got themselves tested in say last 24 hours, or 1 week, or 4 weeks etc. Any other parameter or a combination thereof is well within the scope of the present invention.

In a further exemplary and non-limiting limitation, when a user arrives at an establishment, he/she can check-in into the location to transmit his/her own health/medical profile so that the establishment can then decide whether or not to allow entry of said user based on their defined thresholds. In order to maintain privacy, the user can also configure the system such that only his/her initials or a unique code is transmitted to the establishment along with the syndromic profile so that the unique code/identifier can be presented at the time of entry and allow the establishment to decide on whether or not to allow entry.

In an exemplary aspect, the proposed system can be configured to receive, at a first user's device that is intending to enter into an establishment, based on geographic information of devices of a second set of users that are within the establishment to determine the syndromic profile of such users. For example, the system can use different wireless radios present on mobile devices to gather different types of geographical information. Some radios include cellular, Bluetooth® (trademark of Bluetooth Sig Inc.), global positioning system (or GPS), Wi-Fi, near field communications (or NFC), and other radios. Different sources of location information can result in different levels of geographical specificity.

In an aspect, the proposed system can be configured to, on an ongoing basis, collect, analyze, interpret, and disseminate syndromic data. The proposed system can gather pre-screening, screening, diagnostics and vaccination data, and process said data using a set of executable logic instructions to analyze, interpret and determine the result (Positive or Negative) of a symptom/disease. When an individual visits a location, the system anonymizes and ties the result/outcome of the system to a location's geo code and distributes the syndromic data of establishments and facility, in real-time to all subscribers of the system. In an aspect, the proposed system can dynamically create pre-screening set of questions that a user can be required to respond to as Yes/No. In creating the set of questions, an individual's point of origin syndromic alerts and destination syndromic screening requirements can be considered.

Examples—Yellow Fever syndrome—This infection is most common in areas of Africa and South America, affecting travelers to and from those areas. Accordingly, an individual traveling from Africa to the US will get questions pertinent to Yellow Fever in addition to COVID-19 related questions. Whereas an individual traveling to the US from Canada will get a set of questions pertinent to only COVID-19 screening. At a local level, a restaurant with indoor seating can have a different set of questions for individuals visiting from high-risk areas vis-à-vis a restaurant with outdoor seating allowing individuals from low-risk areas. A cinema hall or an Airport boarding gate may have additional questions along with the requirement of providing a negative test certificate that has not expired.

In an aspect, the proposed system allows business establishments, educational institutions and healthcare facilities to collect vitals at the point of entry (POE) and through Random screening. The systems' integrated devices like wireless contactless thermometers, SPO2, Blood Pressure, Weight Scales, ECG/EKGs, etc. enable real-time screening of individuals visiting these facilities. The result/outcome of the proposed system can use this vital data to analyze and interpret results (positive or negative) and tie it to the geo code of the establishment or facility. In yet another aspect, the proposed system can aggregate data from lab (PCR or Rapid Test), imaging (X-Ray, Ultrasound, CT and MRI), cardiology (Echocardiogram, Pacemaker, Holter and EKG) and Pulmonary (PFT and Stress SPO2) systems and uses it in analysing and interpreting the result. The expiry of the Lab results (PCR and Rapid test) and other diagnostic result dates are also considered in interpreting the result. In yet another aspect of the present proposed system, in addition to the currently active test certification, if a user conducts one or more vital sign tests, e.g. temperature test or SpO2 test, or Spirometer test etc., such test result if cautionary, may influence for an immediate expiration. For example, if a user was certified negative for COVID-19 on Tuesday, and the certificate expires week from the day, any vital sign test resulting positive, should invalidate the certificate or at the least be flagged as requirement for recertification. It is quite possible that the high temperature or other symptom may have subsided during spot test at the entry point and person is allowed entry as asymptomatic. So, the certificate expiration process needs to be more intelligently aware of the interim vital sign tests conducted between otherwise static expiration period.

In an aspect, a multi-level algorithm can be configured to determine positive or negative outcome, wherein the top level (Level-1), for instance, can be of Vaccination, below which can be a Level-2 for Testing, followed by Level-3 of Screening, and Level-4 of Pre-screening questions. In a further aspect, expiry can be considered for any or a combination of levels, for instance, expiry for Level 1 can be 12-24 months based on manufacturer recommendation, for Level 2 can be 3-30 days based on type of test (PCR or RT PCR), for Level-3 can be 1 day (Screening), and for Level-4 can be 1 day (Pre-Screening). In yet another aspect, while determining the outcome, date of every level timeline can be considered for assessment of the user profile.

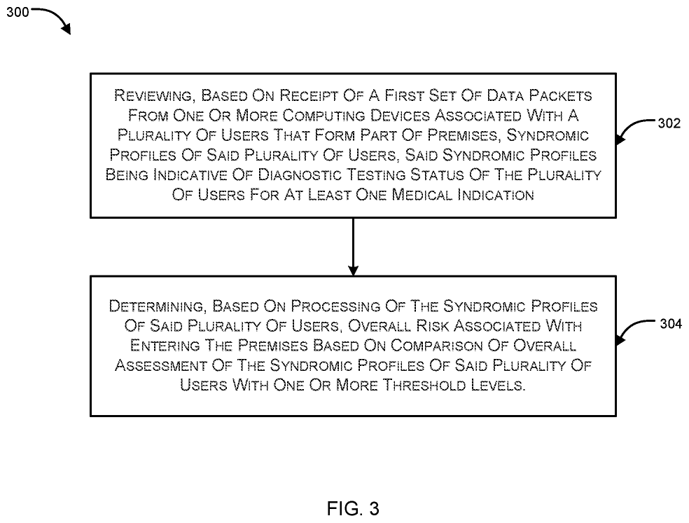

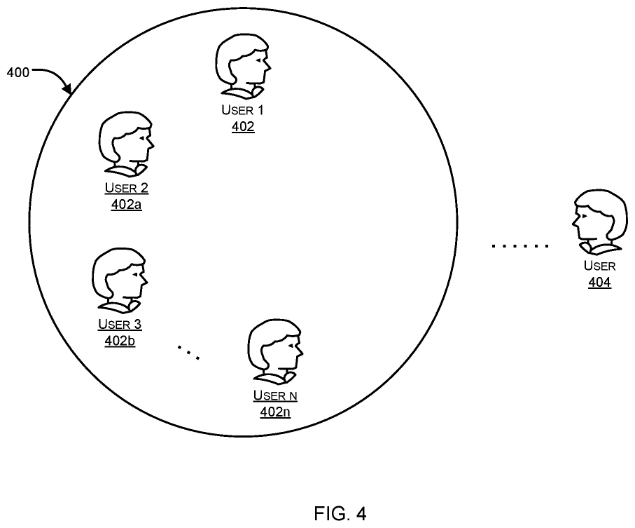

In an aspect, outcome/result of the proposed system can be made available at any time, on-demand, to an individual checking the health of a business establishment or a facility. At the point of entry, the outcome result can be available to a visitor when checking in. At frequent intervals, the outcome can continue to update visitors about the health of the current location. FIG. 3 illustrates an exemplary flow diagram 300 showing exemplary implementation of the proposed invention. The method, at step 302, reviews, based on receipt of a first set of data packets from one or more computing devices associated with a plurality of users that form part of premises, syndromic profiles of said plurality of users, said syndromic profiles being indicative of diagnostic testing status of the plurality of users for at least one medical indication. At step 304, the method determines, based on processing of the syndromic profiles of said plurality of users, overall risk associated with entering the premises based on comparison of overall assessment of the syndromic profiles of said plurality of users with one or more threshold levels. FIG. 4 illustrates an exemplary representation showing premises 400 having a set of users/occupants 402 and a new user 404 intending to enter the premises 404 and hence wishes to understand the risk of entering based on assessment of the testing results/syndromic profile of the current users/occupants of the premises 400. The present disclosure relates to a system for determination of perceived risk of entering a premises, said system comprising: a processor; a memory storing a set of instructions, which when executed by the processor, cause the processor to: review, based on receipt of a first set of data packets from one or more computing devices associated with a plurality of users that form part of premises, syndromic profiles of said plurality of users, said syndromic profiles being indicative of diagnostic testing status of the plurality of users for at least one medical indication; and determine, based on processing of the syndromic profiles of said plurality of users, overall risk associated with entering the premises based on comparison of overall assessment of the syndromic profiles of said plurality of users with one or more threshold levels. In an aspect, the at least one medical indication is selected from any or a combination of Covid-19, a viral infection/disease, and a contagious disease. In another aspect, the first set of data packets are received from the one or more computing devices associated with the plurality of users through an interface that wirelessly receives the first set of data packets from users that have published their diagnostic testing status. In yet another aspect, the one or more computing devices are selected from any or a combination of a laptop, a mobile phone, a smart phone, a wearable device, and a processing unit associated with the respective user. In an aspect, a first user of the plurality of users determine their respective diagnostic testing status through a self-administering test (SAT) kit, said testing status being authenticated by a trained medical service provider that is configured as a trusted authority (TA). In another aspect, syndromic profile of a respective user further indicates vaccination status of the respective user for the at least one medical indication. In another aspect, the syndromic profile of the respective user further indicates when the last test was done, testing history, whether the user contracted the at least one medical indication in the past, travel history of the respective user, and demographic profile of the said user. In another aspect, the first set of data packets are received from such users that have authorized their testing status to be transmitted, and/or whose testing status has been authenticated. In yet another aspect, the first set of data packets are received only from such users that are within a specific range/proximity from the processor. In an embodiment of the invention, the first set of data packets are received in response to a query sent through the processor to the plurality of users that are within defined proximity. In another embodiment, the first set of data packets are received in response to a query sent through the processor remotely to the plurality of users that are identified based on a geo-location specification mentioned in the query. In yet another aspect, the first set of data packets are received from a manager of the premises so as to represent the syndromic profiles of said plurality of users that are currently occupying the premises. In another aspect, the system establishes an enforceable restrictive access for a second user from entering the premises based on determination of the syndromic profile of the respective second user. In another aspect, the system is executable to display, on a display device, through a graphical representation means, any or a combination of the syndromic profiles of said plurality of users and/or the determined risk. In another aspect, the graphical representation is undertaken through any or a combination of a color coding scheme, or granularity region based on region's regulatory body's mandated or enforced characterization, such that when the user hovers around a pin of a desired region, the display presents the graphical representation for the desired region.

It should be understood that the present invention is not limited to any computing device in a specific form factor (e.g., desktop computer form factor), but can include all types of computing devices in various form factors. A user can interface with any computing device, including smartphones, personal computers, laptops, electronic tablet devices, global positioning system (GPS) receivers, portable media players, personal digital assistants (PDAs), other network access devices, and other processing devices capable of receiving or transmitting data.

For example, in a specific implementation, the user/client device can be a smartphone or tablet device, such as the Apple iPhone (e.g., Apple iPhone 6), Apple iPad (e.g., Apple iPad or Apple iPad mini), Apple iPod (e.g, Apple iPod Touch), Samsung Galaxy product (e.g., Galaxy S series product or Galaxy Note series product), Google Nexus devices (e.g., Google Nexus 6, Google Nexus 7, or Google Nexus 9), and Microsoft devices (e.g., Microsoft Surface tablet). Typically, a smartphone includes a telephony portion (and associated radios) and a computer portion, which are accessible via a touch screen display.

There is non-volatile memory to store data of the telephone portion (e.g., contacts and phone numbers) and the computer portion (e.g., application programs including a browser, pictures, games, videos, and music). The smartphone typically includes a camera (e.g., front facing camera or rear camera, or both) for taking pictures and video. For example, a smartphone or tablet can be used to take live video that can be streamed to one or more other devices.

In an exemplary aspect, enclosure houses familiar computer components, some of which are not shown, such as a processor, memory, mass storage devices, and the like. Mass storage devices may include mass disk drives, floppy disks, magnetic disks, optical disks, magneto-optical disks, fixed disks, hard disks, CD-ROMs, recordable CDs, DVDs, recordable DVDs (e.g., DVD-R, DVD+R, DVD-RW, DVD+RW, HD-DVD, or Blu-ray Disc), flash and other nonvolatile solid-state storage (e.g., USB flash drive), battery-backed-up volatile memory, tape storage, reader, and other similar media, and combinations of these. Computer software products may be written in any of various suitable programming languages, such as PHP, ASP, .NET, iOS, Android, Python, Ruby on Rails® (trademark of Hansson, David Heinemeier), PostgreSQL (or Postgres) with PostGis, C, C++, C#, Pascal, Fortran, Perl, Matlab (from MathWorks, www.mathworks.com), SAS, SPSS, JavaScript, AJAX, and Java. The computer software product may be an independent application with data input and data display modules. Alternatively, the computer software products may be classes that may be instantiated as distributed objects. The computer software products may also be component software such as Java Beans (from Sun Microsystems) or Enterprise Java Beans (EJB from Sun Microsystems). The computer software can include database operating using various database implementations, such as MySQL, REDIS, or other database software.

An operating system for the system may be one of the Microsoft Windows® (trademark of Microsoft) family of operating systems (e.g., Windows 95, 98, Me, Windows NT, Windows 2000, Windows XP, Windows XP x64 Edition, Windows Vista, Windows 7, Windows 8, Windows 10, Windows CE, Windows Mobile), Linux, HP-UX, UNIX, Sun OS, Solaris, Mac OS X, Apple iOS, Android, Alpha OS, AIX, IRIX32, or IRIX64. Other operating systems may be used. Microsoft Windows is a trademark of Microsoft Corporation.

Furthermore, the computer may be connected to a network and may interface to other computers using this network. The network may be an intranet, internet, or the Internet, among others. The network may be a wired network (e.g., using copper), telephone network, packet network, an optical network (e.g., using optical fiber), or a wireless network, or any combination of these. For example, data and other information may be passed between the computer and components (or steps) of a system of the invention using a wireless network using a protocol such as Wi-Fi (IEEE standards 802.11, 802.11a, 802.11b, 802.11e, 802.11g, 802.11i, 802.11n, and 802.11ac, and 802.11ad, just to name a few examples), near field communication (NFC), radio-frequency identification (RFID), mobile or cellular wireless (e.g., 2G, 3G, 4G, 3GPP LTE, WiMAX, LTE, Flash-OFDM, HIPERMAN, iBurst, EDGE Evolution, UMTS, UMTS-TDD, 1×RDD, and EV-DO). For example, signals from a computer may be transferred, at least in part, wirelessly to components or other computers.

In an embodiment, with a web browser executing on a computer workstation system, a user accesses a system on the World Wide Web (WWW) through a network such as the Internet. The web browser is used to download web pages or other content in various formats including HTML, XML, text, PDF, and postscript, and may be used to upload information to other parts of the system. The web browser may use uniform resource identifiers (URLs) to identify resources on the web and hypertext transfer protocol (HTTP) in transferring files on the web.

A computer-implemented or computer-executable version or computer program product of the invention may be embodied using, stored on, or associated with a computer-readable medium. A computer-readable medium may include any medium that participates in providing instructions to one or more processors for execution. Such a medium may take many forms including, but not limited to, nonvolatile, volatile, and transmission media. Nonvolatile media includes, for example, flash memory, or optical or magnetic disks. Volatile media includes static or dynamic memory, such as cache memory or RAM. Transmission media includes coaxial cables, copper wire, fiber optic lines, and wires arranged in a bus. Transmission media can also take the form of electromagnetic, radio frequency, acoustic, or light waves, such as those generated during radio wave and infrared data communications.

For example, a binary, machine-executable version, of the software of the present invention may be stored or reside in RANI or cache memory, or on mass storage device 217. The source code of the software of the present invention may also be stored or reside on mass storage device 217 (e.g., hard disk, magnetic disk, tape, or CD-ROM). As a further example, code of the invention may be transmitted via wires, radio waves, or through a network such as the Internet.

It can be appreciated by those versed in the art that the apparatus and system described herein are illustrations of an embodiment of the present disclosure and that they may not be construed as limitations to the scope of the present disclosure.

In an implementation, the system uses geofencing. A geofence is a virtual space corresponding to a real-world geographical (or physical) location. The real-world geographical location tracked by a single geofence can correspond to areas of different sizes. For example, a geofence can include a home, shopping center, school, workplace, city or other location of larger or smaller sizes. A geofence can be established by defining a center-point and a radius distance from the center-point, which determines the overall geographical area covered by the geofence. Usually, the center-point will be the location of interest for the geofence. In another implementation, a geofence can take other shapes, such as a rectangle, square, polygon, or other shape. When a device enters or exits a geofence, a notification is generated. This notification is transmitted to the system for further analysis.

The system can use two types of geofences, individually or in combination: (1) geographic (or physical) geofences and (2) beacon (or proximity) geofencing information. Geographical geofences refers to a location that can be identified as an area on a geographical map. Some sources of geographical geofences used by the system include GPS, cellular tower data, Wi-Fi access point, or any combination of these. Beacon geofencing information refers to a location that can be identified as an area near a physical device or beacon. Some sources of proximity geofences usable by the system include Bluetooth, NFC, Wi-Fi, or other radios. For this application, the term geofencing refers to one of or both of these types of geofences (e.g., proximity and geographic geofencing).

Geofencing can use one or more geographic information sources, in combination or separately. For example, when using GPS, the location of a device can be determined accurately up to several meters (e.g., five meters to fifteen meters). However, GPS oftentimes will not work indoors. When using a Wi-Fi access point, the location of a device can be determined accurately up to a router's maximum range (e.g., twenty meters to two hundred meters). When using cellular towers, the location of a device can be determined up to several thousand meters (e.g., five hundred meters to ten kilometers). This will depend on how many cellular antennas are in a given area. For areas with a greater density of antennas, the accuracy using this method is higher. When using Bluetooth, the location of a device can be determined to within less than a meter (e.g., using triangulation).

Since using these sources of geographical information can be battery intensive and vary in accuracy, the system is able to use different sources of geographical information in combination or separately depending on different situations. For example, while GPS is oftentimes the most accurate, it also results in a high battery drain. So, geofencing can use Wi-Fi or cellular tower information instead. Also, to conserve battery power, the system does not require real-time geographical information all the time. For example, the system works with limited geographical information, such information taken every given interval of time (e.g., one, two, five, ten, fifteen minutes or one, two, three, four hours, or any amount of time), or according to an operating system specified method, an application specified method, or other methods as discussed in this application.

In an implementation, the system uses geofencing abilities defined by a device's operating system. For example, the system does not directly implement methods to retrieve geofencing or location information from a device, but accesses the information through functions built into the device's operating system. The location information retrieved using this method is agnostic; this means that the operating system for the device reports to the system location information that can come from any of the geographical information sources as described elsewhere in this application. Different operating systems can impose different limitations on the number of geofences that can be monitored by operating systems. For example, for Apple's iOS operating system, applications are limited to monitoring twenty geofences per application installed on a device at a given time. On the other hand, for Google's Android℠ (service mark of Google Inc.) operating system, applications can monitor one hundred geofences per application installed on a device at a given time. These limitations assist the different operating systems to control battery consumption and can change according to different technological trends (e.g., improved battery capacity, more efficient components, or other trends).

In additional aspects, geofences can be used on a location-aware mobile device to detect when a user of the mobile device enters a specific location, such as a specific retail store. Geofences can be defined in terms of Global Positioning System (GPS) coordinates (e.g., latitude and longitude) combined with a radius measured in meters or feet, for example. Alternatively, geofences can also be defined according a series of GPS coordinates defining a bounding box. In yet other examples, a geofence can be any geometric shape defined by a mathematical formula and anchored by a GPS coordinate. Mobile devices, such as an iPhone (from Apple, Inc. of Cupertino, Calif.) can monitor a number of geofences at a given time. Additionally, applications running on a mobile device commonly can update monitored geofences when the application is opened by a user (or at least active in memory on the mobile device).

### Modules, Components and Logic

Certain embodiments are described herein as including logic or a number of components, modules, or mechanisms. Modules may constitute either software modules (e.g., code embodied on a machine-readable medium or in a transmission signal) or hardware modules. A hardware module is a tangible unit capable of performing certain operations and may be configured or arranged in a certain manner. In example embodiments, one or more computer systems (e.g., a standalone, client or server computer system or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired) or temporarily configured (e.g., programmed) to operate in a certain manner and/or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods described herein may be at least partially processor-implemented. For example, at least some of the operations of a method may be performed by one or processors or processor-implemented modules. The performance of certain of the operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of locations.

The one or more processors may also operate to support performance of the relevant operations in a “cloud computing” environment or as a “software as a service” (SaaS). For example, at least some of the operations may be performed by a group of computers (as examples of machines including processors), with these operations being accessible via a network (e.g., the Internet) and via one or more appropriate interfaces (e.g., APIs).

### Electronic Apparatus and System

Example embodiments may be implemented digital electronic circuitry, or in computer hardware, firmware, software, or in combinations of them. Example embodiments may be implemented using a computer program product, for example, a computer program tangibly embodied in an information carrier, for example, in a machine-readable medium for execution by, or to control the operation of data processing apparatus, for example, a programmable processor, a computer, or multiple computers.

A computer program can be written in any form of programming language, including compiled or interpreted languages, and it can be deployed in any form, including as a stand-alone program or as a module, subroutine, or other unit suitable for use in a computing environment. A computer program can be deployed to be executed on one computer or on multiple computers at one site or distributed across multiple sites and interconnected by a communication network.

In example embodiments, operations may be performed by one or more programmable processors executing a computer program to perform functions by operating on input data and generating output. Method operations can also be performed by, and apparatus of example embodiments may be implemented as, special purpose logic circuitry (e.g., a FPGA or an ASIC).

The computing system can include clients and servers. A client and server are generally remote from each other and typically interact through a communication network. The relationship of client and server arises by virtue of computer programs running on the respective computers and having a client-server relationship to each other. In embodiments deploying a programmable computing system, it will be appreciated that both hardware and software architectures require consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware may be a design choice. Below are set out hardware (e.g., machine) and software architectures that may be deployed, in various example embodiments.

### Example Machine Architecture and Machine-Readable Medium

Aspects of the present disclosure pertain to a computer system within which instructions, for causing the machine to perform any one or more of the methodologies discussed herein, may be executed. In alternative embodiments, the machine operates as a standalone device or may be connected (e.g., networked) to other machines. In a networked deployment, the machine may operate in the capacity of a server or a client machine in server-client network environment, or as a peer machine in a peer-to-peer (or distributed) network environment. The machine may be a personal computer (PC), a tablet PC, a set-top box (STB), a PDA, a cellular telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

The example computer system includes a processor (e.g., a central processing unit (CPU), a graphics processing unit (GPU) or both), a main memory and a static memory, which communicate with each other via a bus. The computer system may further include a video display unit (e.g., a liquid crystal display (LCD) or a cathode ray tube (CRT)). The computer system also includes an alphanumeric input device (e.g., a keyboard), a user interface (UI) navigation device (e.g., a mouse), a disk drive unit, a signal generation device (e.g., a speaker) and a network interface device.

### Machine-Readable Medium

The disk drive unit includes a machine-readable medium on which is stored one or more sets of instructions and data structures (e.g., software) embodying or used by any one or more of the methodologies or functions described herein. The instructions may also reside, completely or at least partially, within the main memory, static memory, and/or within the processor during execution thereof by the computer system, the main memory and the processor also constituting machine-readable media.

While the machine-readable medium is shown in an example embodiment to be a single medium, the term “machine-readable medium” may include a single medium or multiple media (e.g., a centralized or distributed database, and/or associated caches and servers) that store the one or more instructions or data structures. In the context of the present invention, the machine-readable media can also be explanted or implanted in the user's body from which the relevant user health information or vaccination certificates can be retrieved and exchanged with the user's device. The explanted/implanted media may also be an active device periodically monitoring growth of viral culture and/or changing vital signs. The term “machine- readable medium” shall also be taken to include any tangible medium that is capable of storing, encoding or carrying instructions for execution by the machine and that cause the machine to perform any one or more of the methodologies of the present invention, or that is capable of storing, encoding or carrying data structures used by or associated with such instructions. The term “machine-readable medium” shall accordingly be taken to include, but not be limited to, solid-state memories, and optical and magnetic media. Specific examples of machine-readable media include non-volatile memory, including by way of example, semiconductor memory devices (e.g., Erasable Programmable Read-Only Memory (EPROM), Electrically Erasable Programmable Read-Only Memory (EEPROM)) and flash memory devices; magnetic disks such as internal hard disks and removable disks; magneto-optical disks; and CD-ROM and DVD-ROM disks.

### Transmission Medium

The instructions may further be transmitted or received over a communications network using a transmission medium. The instructions may be transmitted using the network interface device and any one of a number of well-known transfer protocols (e.g., HTTP). Examples of communication networks include a LAN, a WAN, the Internet, mobile telephone networks, Plain Old Telephone (POTS) networks, and wireless data networks (e.g., Wi-Fi and WiMax networks). The term “transmission medium” shall be taken to include any intangible medium that is capable of storing, encoding or carrying instructions for execution by the machine, and includes digital or analog communications signals or other intangible media to facilitate communication of such software.

### Non-limiting Embodiments

Although the present invention has been described with reference to specific example embodiments, it will be evident that various modifications and changes may be made to these embodiments without departing from the broader spirit and scope of the invention. Accordingly, the specification and drawings are to be regarded in an illustrative rather than a restrictive sense.

Although an embodiment has been described with reference to specific example embodiments, it will be evident that various modifications and changes may be made to these embodiments without departing from the broader spirit and scope of the invention. Accordingly, the specification and drawings are to be regarded in an illustrative rather than a restrictive sense. The accompanying drawings that form a part hereof, show by way of illustration, and not of limitation, specific embodiments in which the subject matter may be practiced. The embodiments illustrated are described in sufficient detail to enable those skilled in the art to practice the teachings disclosed herein. Other embodiments may be used and derived therefrom, such that structural and logical substitutions and changes may be made without departing from the scope of this disclosure. This Detailed Description, therefore, is not to be taken in a limiting sense, and the scope of various embodiments is defined only by the appended claims, along with the full range of equivalents to which such claims are entitled.

Such embodiments of the inventive subject matter may be referred to herein, individually and/or collectively, by the term “invention” merely for convenience and without intending to voluntarily limit the scope of this application to any single invention or inventive concept if more than one is in fact disclosed. Thus, although specific embodiments have been illustrated and described herein, it should be appreciated that any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

It should be apparent to those skilled in the art that many more modifications besides those already described are possible without departing from the inventive concepts herein. The inventive patent matter, therefore, is not to be restricted except in the spirit of the appended claims. Moreover, in interpreting both the specification and the claims, all terms should be interpreted in the broadest possible manner consistent with the context. In particular, the terms “includes” and “including” should be interpreted as referring to elements, components, or steps in a non-exclusive manner, indicating that the referenced elements, components, or steps may be present, or utilized, or combined with other elements, components, or steps that are not expressly referenced. Where the specification claims refer to at least one of something selected from the group consisting of A, B, C . . . and N, the text should be interpreted as requiring any usable combination of one or more elements from the group, not necessarily A plus B plus C . . . plus N, etc. The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognise that the embodiments herein can be practised with modification within the spirit and scope of the appended claims.

While the foregoing describes various embodiments of the invention, other and further embodiments of the invention may be devised without departing from the basic scope thereof. The scope of the invention is determined by the claims that follow. The invention is not limited to the described embodiments, versions or examples, which are included to enable a person having ordinary skill in the art to make and use the invention when combined with information and knowledge available to the person having ordinary skill in the art.

1. A system for determination of perceived risk of entering a premises, said system comprising:
a processor;
a memory storing a set of instructions, which when executed by the processor, cause the processor to:
review, based on receipt of a first set of data packets from one or more computing devices associated with a plurality of users that form part of premises, syndromic profiles of said plurality of users, said syndromic profiles being indicative of diagnostic testing status of the plurality of users for at least one medical indication; and
determine, based on processing of the syndromic profiles of said plurality of users, overall risk associated with entering the premises based on comparison of overall assessment of the syndromic profiles of said plurality of users with one or more threshold levels.

2. The system as claimed in claim 1, wherein the at least one medical indication is selected from any or a combination of Covid-19, a viral infection/disease, and a contagious disease.

3. The system as claimed in claim 1, wherein the first set of data packets are received from the one or more computing devices associated with the plurality of users through an interface that wirelessly receives the first set of data packets from users that have published their diagnostic testing status.

4. The system as claimed in claim 1, wherein the one or more computing devices are selected from any or a combination of a laptop, a mobile phone, a smart phone, a wearable device, and a processing unit associated with the respective user.

5. The system as claimed in claim 1, wherein a first user of the plurality of users determine their respective diagnostic testing status through a self-administering test (SAT) kit, said testing status being authenticated by a trained medical service provider that is configured as a trusted authority (TA).

6. The system as claimed in claim 1, wherein syndromic profile of a respective user further indicates vaccination status of the respective user for the at least one medical indication.

7. The system as claimed in claim 1, wherein the syndromic profile of the respective user further indicates when the last test was done, testing history, whether the user contracted the at least one medical indication in the past, travel history of the respective user, and demographic profile of the said user.

8. The system as claimed in claim 1, wherein the first set of data packets are received from such users that have authorized their testing status to be transmitted, and/or whose testing status has been authenticated.

9. The system as claimed in claim 1, wherein the first set of data packets are received only from such users that are within a specific range/proximity from the processor.

10. The system as claimed in claim 1, wherein the first set of data packets are received in response to a query sent through the processor to the plurality of users that are within defined proximity.

11. The system as claimed in claim 1, wherein the first set of data packets are received in response to a query sent through the processor remotely to the plurality of users that are identified based on a geo-location specification mentioned in the query.

12. The system as claimed in claim 1, wherein the first set of data packets are received from a manager of the premises so as to represent the syndromic profiles of said plurality of users that are currently occupying the premises.

13. The system as claimed in claim 1, wherein the system establishes an enforceable restrictive access for a second user from entering the premises based on determination of the syndromic profile of the respective second user.

14. The system as claimed in claim 1, wherein the system is executable to display, on a display device, through a graphical representation means, any or a combination of the syndromic profiles of said plurality of users and/or the determined risk.

15. The system as claimed in claim 1, wherein the graphical representation is undertaken through any or a combination of a color coding scheme, or granularity region based on region's regulatory body's mandated or enforced characterization, such that when the user hovers around a pin of a desired region, the display presents the graphical representation for the desired region.

16. A method for determination of perceived risk of entering a premises, said method comprising the steps of:
reviewing, based on receipt of a first set of data packets from one or more computing devices associated with a plurality of users that form part of premises, syndromic profiles of said plurality of users, said syndromic profiles being indicative of diagnostic testing status of the plurality of users for at least one medical indication; and
determining, based on processing of the syndromic profiles of said plurality of users, overall risk associated with entering the premises based on comparison of overall assessment of the syndromic profiles of said plurality of users with one or more threshold levels.

17. The method as claimed in claim 16, wherein the first set of data packets are received from the one or more computing devices associated with the plurality of users through an interface that wirelessly receives the first set of data packets from users that have published their diagnostic testing status.

18. The method as claimed in claim 16, wherein syndromic profile of a respective user further indicates vaccination status of the respective user for the at least one medical indication, and wherein the syndromic profile of the respective user further indicates when the last test was done, testing history, whether the user contracted the at least one medical indication in the past, travel history of the respective user, and demographic profile of the said user.

19. The method as claimed in claim 16, wherein the first set of data packets are received from such users that have authorized their testing status to be transmitted, and/or whose testing status has been authenticated.

20. The method as claimed in claim 16, wherein the first set of data packets are received only from such users that are within a specific range/proximity from the processor.
